(19) United States
(12) Patent Application Publication
(10) Pub. No.: US 2009/0082841 A1
(43) Pub. Date: Mar. 26, 2009

(54) APPARATUS FOR SECURING STENT BARBS

(75) Inventors: Isaac J. Zacharias, Pleasanton, CA (US); Maurice Marthaler, Santa Rosa, CA (US)

Correspondence Address: GRANT ANDERSON LLP, GRANT ANDERSON LLP C/O PORTFOLIOIP, P.O. BOX 52050, MINNEAPOLIS, MN, 55402, US

(73) Assignee: BOSTON SCIENTIFIC CORPORATION, Maple Grove, MN (US)

(21) Appl. No.: 11/861,739

(22) Filed: Sep. 26, 2007

(51) Int. Cl. A61F 2/84; A61F 2/82

(52) U.S. Cl. 623/1.11; 623/1.15; 623/1.16

(57) ABSTRACT

A stent system comprising a stent body. At least one barb extends from the stent body and is configured such that a free end thereof is biased to extend radially outward from the stent body. A retaining mechanism is positioned to engage the at least one barb when the stent body is in a compressed state and retain the at least one barb in a tucked position relative to the stent body.

### BACKGROUND OF THE INVENTION

This invention relates generally to endoluminal devices, particularly stents and grafts for placement in an area of a body lumen that has been weakened by damage or disease, such as an aneurysm of the abdominal aorta, and more particularly to devices having characteristics that enhance affixation of the devices to the body lumen.

Medical devices for placement in a human or other animal body are well known in the art. One class of medical devices comprises endoluminal devices such as stents, stent-grafts, filters, coils, occlusion baskets, valves, and the like. A stent typically is an elongated device used to support an intraluminal wall. In the case of a stenosis, for example, a stent provides an unobstructed conduit through a body lumen in the area of the stenosis. Such a stent may also have a prosthetic graft layer of fabric or covering lining the inside and/or outside thereof. A covered stent is commonly referred to in the art as an intraluminal prosthesis, an endoluminal or endovascular graft (EVG), a stent-graft, or endograft.

An endograft may be used, for example, to treat a vascular aneurysm by removing or reducing the pressure on a weakened part of an artery so as to reduce the risk of rupture. Typically, an endograft is implanted in a blood vessel at the site of a stenosis or aneurysm endoluminally, i.e. by so-called “minimally invasive techniques” in which the endograft, typically restrained in a radially compressed configuration by a sheath, crocheted or knit web, catheter or other means, is delivered by an endograft delivery system or “introducer” to the site where it is required. The introducer may enter the vessel or lumen from an access location outside the body, such as purcutaneously through the patient's skin, or by a “cut down” technique in which the entry vessel or lumen is exposed by minor surgical means. The term “proximal” as used herein refers to portions of the endograft, stent or delivery system relatively closer to the end outside of the body, whereas the term “distal” is used to refer to portions relatively closer to the end inside the body.

After the introducer is advanced into the body lumen to the endograft deployment location, the introducer is manipulated to cause the endograft to be deployed from its constrained configuration, whereupon the stent is expanded to a predetermined diameter at the deployment location, and the introducer is withdrawn. Stent expansion typically is effected by spring elasticity, balloon expansion, and/or by the self-expansion of a thermally or stress-induced return of a memory material to a pre-conditioned expanded configuration.

Among the many applications for endografts is that of deployment in lumen for repair of an aneurysm, such as a thorasic aortic aneurysm (TAA) or an abdominal aortic aneurysm (AAA). An AAA is an area of increased aortic diameter that generally extends from just below the renal arteries to the aortic bifurcation and a TAA most often occurs in the descending thoracic aorta. AAA and TAA generally result from deterioration of the arterial wall, causing a decrease in the structural and elastic properties of the artery. In addition to a loss of elasticity, this deterioration also causes a slow and continuous dilation of the lumen.

The standard surgical repair of AAA or TAA is an extensive and invasive procedure typically requiring a week long hospital stay and an extended recovery period. To avoid the complications of the surgical procedure, practitioners commonly resort to a minimally invasive procedure using an endoluminal endograft to reinforce the weakened vessel wall, as mentioned above. At the site of the aneurysm, the practitioner deploys the endograft, anchoring it above and below the aneurysm to relatively healthy tissue. The anchored endograft diverts blood flow away from the weakened arterial wall, minimizing the exposure of the aneurysm to high pressure.

Intraluminal stents for repairing a damaged or diseased artery or to be used in conjunction with a graft for delivery to an area of a body lumen that has been weakened by disease or damaged, such as an aneurysm of the thorasic or abdominal aorta, are well established in the art of medical science. Intraluminal stents having barbs, hooks, or other affixation means to secure the stents to the wall of the lumen in which they are to be deployed are also well known in the art.

While barbed and the like stents are advantageous in anchoring the device, an improved system for retaining and releasing stent barbs is desired.

### SUMMARY OF THE INVENTION

In one aspect, the invention provides a stent system comprising a stent body. At least one barb extends from the stent body and is configured such that a free end thereof is biased to extend radially outward from the stent body. A retaining mechanism is positioned to engage the at least one barb when the stent body is in a compressed state and retain the at least one barb in a tucked position relative to the stent body.

In another aspect, the invention provides a stent delivery system comprising a stent body. At least one barb extends from stent body and is configured such that a free end thereof is biased to extend radially outward from the stent body. A support is positioned at least partially within the stent body, said support including a retaining mechanism positioned to engage the at least one barb when the stent body is in a compressed state and retain the at least one barb in a tucked position relative to the stent body.

In another aspect, the invention provides a stent a plurality of struts. A barb extends from at least one of the struts and is configured such that a free end thereof is biased to extend radially outward from the strut. A retaining mechanism is positioned to engage the barb when the stent is in a compressed state and retain the barb in a tucked position relative to the stent, wherein the retaining mechanism comprises a shoulder defined between two portions of at least one strut.

Other aspects and advantages of the present invention will be apparent from the detailed description of the invention provided hereinafter.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 is an isometric view of a bushing retainer mechanism in accordance with a first embodiment of the present invention.

FIG. 2 is a flat pattern of a stent incorporating the bushing retainer mechanism of FIG. 1.

FIG. 3 is an isometric view of a bushing retainer mechanism that is an alternative embodiment of the present invention.

FIG. 4 is an isometric view of a bushing retainer mechanism that is another alternative embodiment of the present invention.

FIG. 5 is a top plan view of a stent delivery system incorporating the bushing retainer mechanism of FIG. 3.

FIG. 6 is an expanded view of one of the bushing retainer mechanisms of FIG. 5.

FIG. 7 is a front plan view of a portion of a stent incorporating an alternative retainer mechanism in accordance with the invention.

FIG. 8 is a rear plan view of a portion of a stent incorporating an alternative retainer mechanism in accordance with the invention.

FIG. 9 shows a flat pattern of a portion of the stent of FIG. 7.

FIG. 10 is a cross-sectional view along the line 10-10 in FIG. 9.

FIG. 11 is a cross-sectional view along the line 11-11 in FIG. 9.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

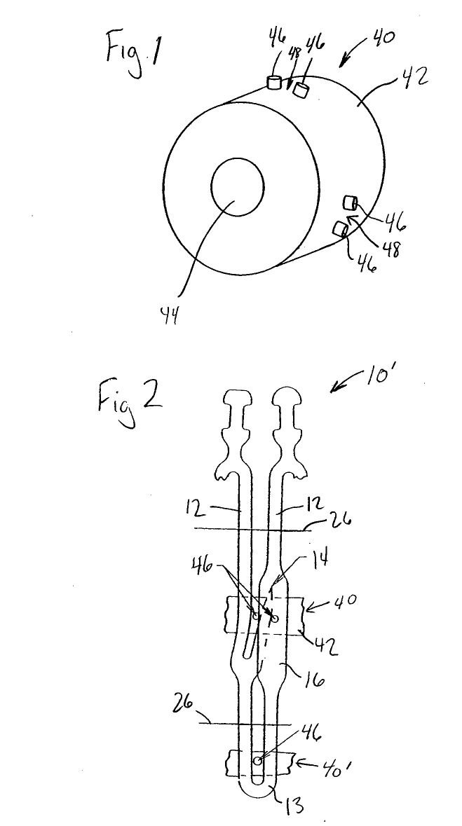

Referring to FIGS. 1-2, a retainer mechanism 40 that is a first embodiment of the present invention is illustrated. The retainer mechanism 40 includes a generally cylindrical bushing body 42. While the bushing body 42 is illustrated as cylindrical, it is not limited to such and may have other configurations. The bushing body 42 includes a through bore 44 configured to receive a delivery catheter or guidewire chassis (not shown) of a stent-graft delivery system. The outer surface of the bushing body 42 includes pairs of radially extending pins 46. Each pair of pins 46 defines a barb receiving space 48 therebetween. The bushing body 42 and the pins 46 may be manufactured from a hard material, for example, polyimide, PEEK or polyurethane, or a softer material, for example, urethanes or silicone, such that the barbs 14 can be compressed within the receiving space 48 and into the surface of the bushing body 42 for increased stability.

FIG. 2 illustrates an illustrative stent 10′ positioned relative to the retainer mechanism 40. The bushing body 42 is axially positioned along the delivery system such that the barbs 14 align with and are received in the receiving space 48 between a respective pair of pins 46. The pins 46 are circumferentially aligned with a respective tuck pad 16 or strut 12 such that the barb 14 received in a receiving space 48 is maintained under the tuck pad 16 or strut 12. Since a pin 46 is provided on each lateral side of the barb 14, the pins 46 will maintain the barb 14 in proper lateral alignment even if the barb lateral angle α is not maintained to the highest tolerances.

The pins 46 have a radial height that is approximately one half of the thickness of the struts 12. As such, the pins 46 do not interfere with the compression of the stent. If the retaining mechanism is manufactured from a softer materials, the bushing body 42 can compress and relieve some of the added thickness of the tucked barb 14.

While the preferred retaining mechanism 40 has the pins 46 in pairs, such is not required and the pins 46 can be grouped individually or in groups of more than two. As illustrated in FIG. 2, a retaining mechanism 40′ with a single pin 46 is provided adjacent an end of the stent 10′ to provide a crown 13 locating feature. Additionally, while the bushing body 42 is illustrated as extending a short axial distance adjacent the barb 14, the body 42 may have a longer axial length. For example, the bushing body 42 may be sufficiently long to extend under one or both belt axial positions such that the belts can be attached to the retaining member 40. Other shapes and configurations of the bushing body 42 and the pins 46 are within the scope of the present invention.

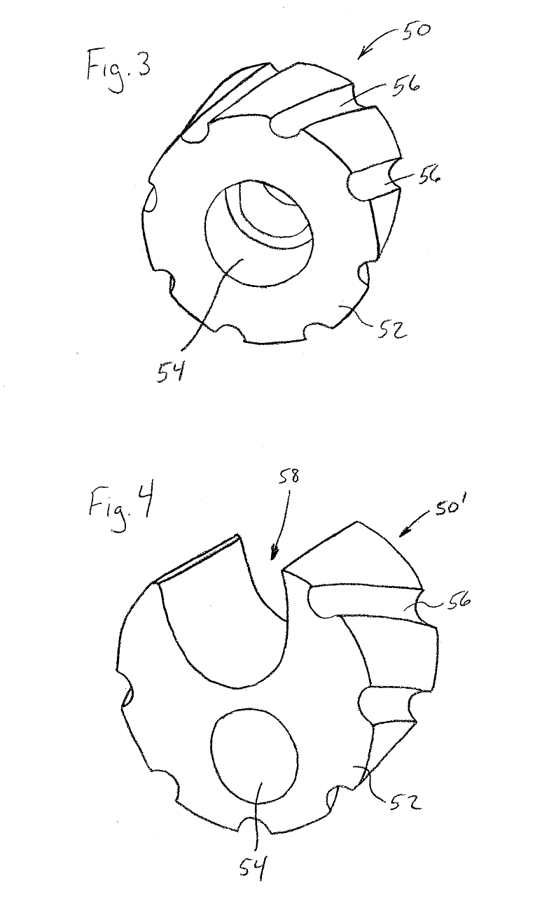

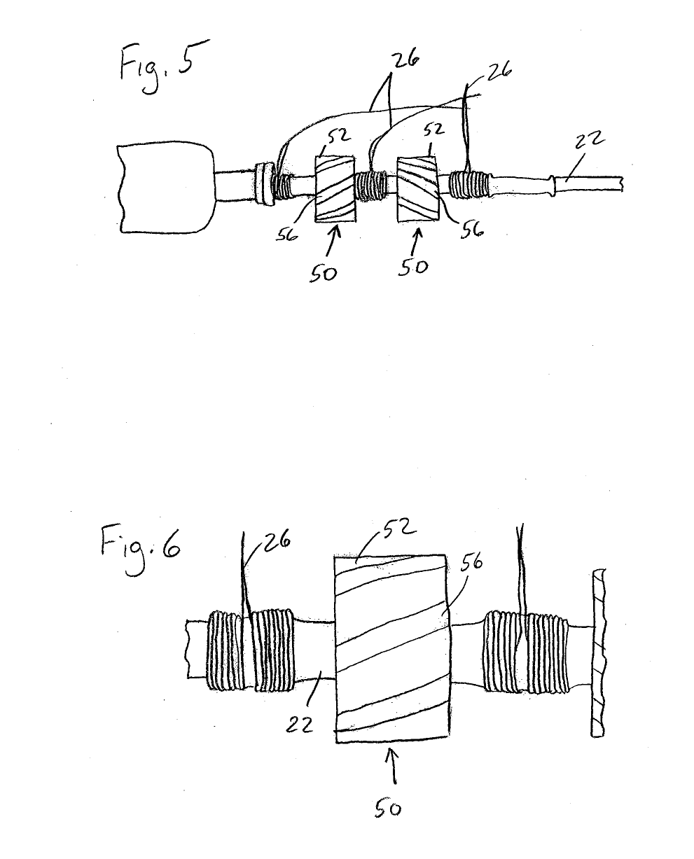

Referring to FIGS. 3 and 5-6, a retaining mechanism 50 that is an alternative embodiment of the present invention will be described. The retaining mechanism 50 is similar to the previous embodiment and includes a bushing body 52 with a through bore 54 configured to receive a guidewire chassis 22 of a delivery system as illustrated in FIGS. 5 and 6. While the retaining mechanism 50 may be secured to the guidewire chassis 22, such is not required and freedom of the retaining mechanism 50 may allow for greater flexibility and alignment. The retaining mechanism 50′ illustrated in FIG. 4 is substantially the same as in the present embodiment but includes a secondary through passage 58. The secondary through passage 58 facilitates passage of additional delivery system items, for example, such as when the retaining mechanism 50′ is used with a distal stent.

Both of the retaining mechanisms 50, 50′ include a plurality of helical slots 56 formed about the outer surface of the bushing body 52. Each slot is configured to receive a barb 14 when the stent 10 is compressed via the belts 26. The helical nature of the slots 56 corresponds with the laying direction of the tucked barbs 14. The slots 56 may have other configurations to accommodate barbs 14 having different configurations. The slots 56 receive the tucked barbs 14 and retain them in the tucked position, aligned with a corresponding strut or tuck pad. Additionally, since the slots 56 are recessed into the bushing body 52, the tucked barbs 14 do not add to the radial size of the compressed stent. As seen in FIG. 5, multiple retaining mechanisms 50 may be utilized with a delivery system. The direction of the slots 56 for the two retaining mechanisms 50 is opposite such that they accommodate barbs 14 extending in opposite directions.

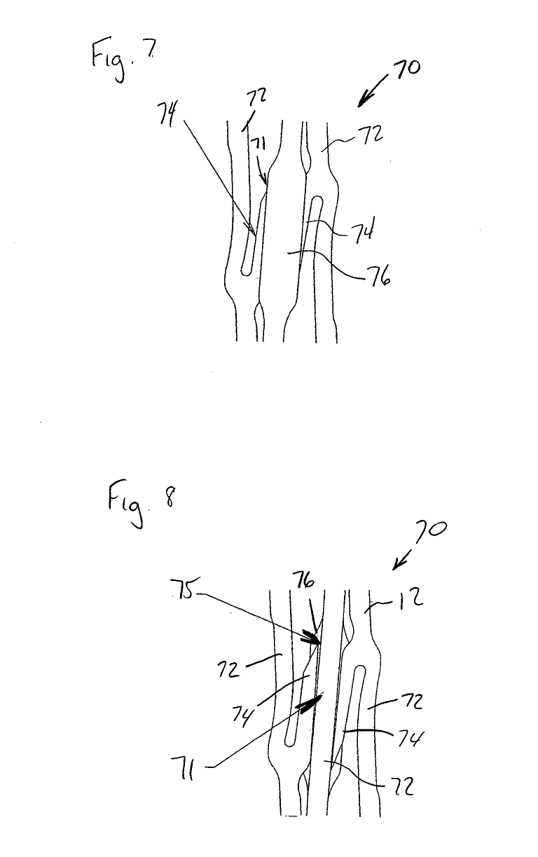

Referring to FIGS. 7-11, a retaining mechanism 71 that is another alternative embodiment of the present invention is shown. The retaining mechanism 71 is formed integrally with the stent 70, as opposed to being accommodated on the delivery system as in the previous embodiments. The retaining mechanism 71 is defined by the stent struts 72 and the associated reduced thickness tuck pads 76.

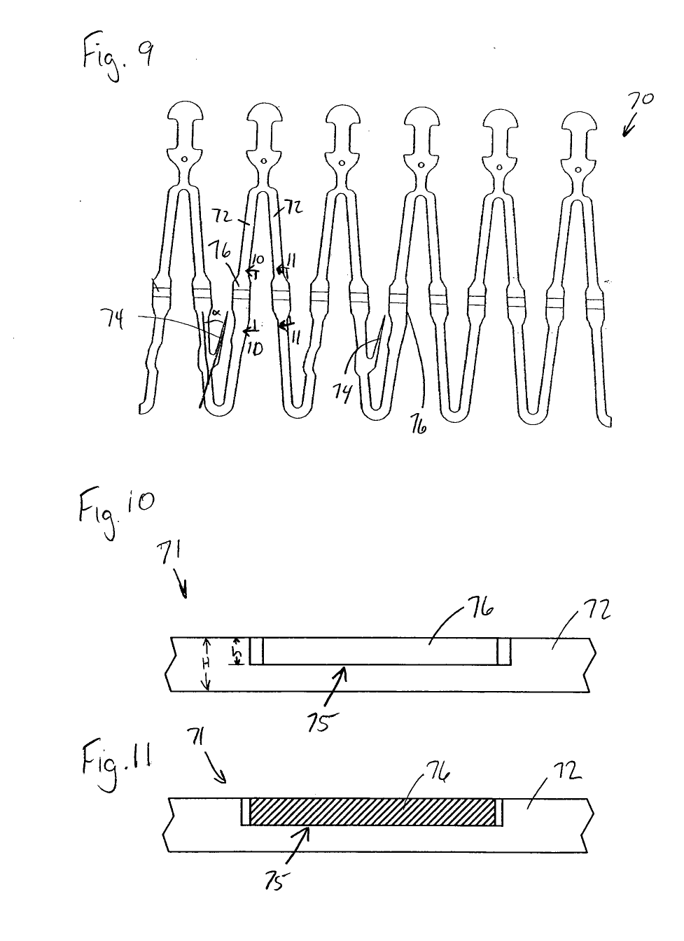

Referring to FIGS. 10 and 11, each tuck pad 76 has a radial height h that is approximately one-half or less the radial height of the corresponding strut 72. As such, the retaining mechanism 71 is defined by the shoulder 75 defined between the strut 72 and tuck pad 76. Referring to FIGS. 8 and 9, in the compressed state, the barbs 74 are forced against the shoulder 75 of the retaining mechanism 71. The risk of the barb 74 overextending past the tuck pad or strut is reduced since the shoulder 75 of the retaining mechanism 71 prevents such. As such, the barb lateral angle α can be increased to ensure that the barbs 74 will not back out while not having to worry about overextension. Additionally, since the tuck pads 76 are approximately one-half or less the height of typical tuck pads, they will have a reduced effect on the radial thickness of the compressed stent 70.

What is claimed:

1. A stent system comprising:
a stent body;
at least one barb extending from the stent body and configured such that a free end thereof is biased to extend radially outward from the stent body; and
a retaining mechanism positioned to engage the at least one barb when the stent body is in a compressed state and retain the at least one barb in a tucked position relative to the stent body.

2. The stent system according to claim 1 wherein the retaining mechanism includes a bushing body configured to be supported on a stent delivery system.

3. The stent system according to claim 2 wherein the bushing body includes a radial surface with one or more radial outwardly extending pins.

4. The stent system according to claim 3 wherein the pins are arranged in pairs and define a barb receiving space in between each pair of pins.

5. The stent system according to claim 4 wherein the pins are manufactured from a compressible material such that the barb is compressed within the respective barb receiving space.

6. The stent system according to claim 4 wherein each barb receiving space is circumferentially aligned with a strut or tuck pad of the stent body.

7. The stent system according to claim 3 wherein the at least one barb has a thickness and each pin has a radial thickness approximately one-half the barb thickness.

8. The stent system according to claim 2 wherein a stent restraining belt is supported by the bushing body.

9. The stent system according to claim 2 wherein the bushing body includes a secondary through passage.

10. The stent system according to claim 2 wherein the bushing body includes a plurality of surface slots configured to receive a corresponding barb.

11. The stent system according to claim 10 wherein each of the slots extends helically.

12. The stent system according to claim 11 wherein the delivery system supports a secondary retaining mechanism including a plurality of surface slots, and wherein the surface slots of the secondary retaining mechanism extend helically in a direction opposite to the slots of the other retaining mechanism.

13. The stent system according to claim 10 wherein each of the slots is configured to circumferentially align with a strut or tuck pad of the stent body.

14. The stent system according to claim 2 wherein the bushing body is free to rotate relative to the stent delivery system.

15. The stent system according to claim 1 wherein the retaining mechanism is formed integrally with the stent body.

16. The stent system according to claim 15 wherein the retaining mechanism includes a shoulder defined between one of the struts and an associated tuck pad.

17. The stent system according to claim 16 wherein the tuck pad has a radial thickness that is approximately one-half or less of a radial thickness of the associated strut.

18. A stent delivery system comprising:
a stent body;
at least one barb extending from stent body and configured such that a free end thereof is biased to extend radially outward from the stent body; and
a support positioned at least partially within the stent body, said support including a retaining mechanism positioned to engage the at least one barb when the stent body is in a compressed state and retain the at least one barb in a tucked position relative to the stent body.

19. A stent comprising:
a plurality of struts;
a barb extending from at least one of said struts and configured such that a free end thereof is biased to extend radially outward from the strut; and
a retaining mechanism positioned to engage the barb when the stent is in a compressed state and retain the barb in a tucked position relative to the stent, wherein the retaining mechanism comprises a shoulder defined between two portions of at least one said strut.
